(19) United States
(12) Patent Application Publication
(10) Pub. No.: US 2025/0093318 A1
(43) Pub. Date: Mar. 20, 2025

(54) SPIKE TYPE FRESHNESS SENSOR

(71) Applicant: The Kroger Co., Cincinnati, OH (US)

(72) Inventors: Addison Carter, Cincinnati, OH (US); Miriam Kolis, Cincinnati, OH (US); Anastacia Dwyer, Cincinnati, OH (US)

(21) Appl. No.: 18/469,184

(22) Filed: Sep. 18, 2023

(51) Int. Cl. G01N 33/00; G01N 33/12

(52) U.S. Cl. CPC G01N 33/0022; G01N 33/12

(57) ABSTRACT

A freshness sensor includes a cap housing a sensor element and a spike extending away from a lower surface of the cap. The spike defines a cavity in fluid communication with the sensor element and the spike includes an expansion portion configured to expand after the spike is inserted into packaging housing a perishable product, so as to form a hollow in the packaging or perishable product and thereby allow gas exchange between the hollow and the sensor element by way of the cavity.

### TECHNICAL FIELD

The present invention relates to freshness sensors. More particularly, the present invention relates to a freshness sensor that is punctured through packaging and into a perishable product, such as meat.

### BACKGROUND

Retail stores, such as grocery stores and supermarkets, lose a considerable amount of revenue each year as the result of unsold fruits, vegetables, meats, and/or other perishable items that are no longer fresh and, thus, are lost to waste. This issue is compounded when determinations regarding whether perishable items are no longer suitable for sale are applied to all of a particular type of perishable item, such as a pallet received from one supplier.

In order to monitor environmental factors affecting the freshness of perishable items, it is known to rely on qualitative measures, such as observing the color or smell of the perishable item, which are notoriously unreliable and imprecise. Alternatively, freshness of perishable items may be determined quantitatively by colony forming units on the surface of the product that typically happens in the suppliers or product quality laboratories. Typically, these direct methods require lab technicians to perform the tests. While some sensing devices have been developed to replace the qualitative and quantitative methods discussed above, they still suffer from certain limitations.

Accordingly, improved freshness sensors would be highly desirable and beneficial.

### SUMMARY

The present invention includes freshness sensor devices and related methods of using the devices. More particularly, the present invention includes a freshness sensor that is punctured through packaging and into a perishable product, such as meat. In some embodiments, the freshness sensor thereafter creates a hollow in the packaging and/or meat to facilitate gas exchange between the meat and the sensor element to monitor factors relating to the freshness of the meat, including, but not limited to, volatile nitrogen and/or gaseous volatile biogenic amines. In other embodiments, the freshness sensor includes electrodes that measure the bulk phase electrical properties of the meat.

In accordance with one exemplary embodiment, a freshness sensor made in accordance with the present invention includes a cap housing a sensor element and a spike extending away from a lower surface of the cap. The spike includes a cavity defined by the spike and in fluid communication with the sensor element, and an expansion portion. The expansion portion is configured to expand after the spike is inserted into a packaging housing a perishable product and form a hollow in the packaging or perishable product to thereby allow gas exchange between the hollow and the sensor element by way of the cavity.

In some embodiments, the freshness sensor further includes an adhesive layer positioned on the lower surface of the cap.

In some embodiments, the spike has a tapered portion forming a terminal distal end of the spike.

In some embodiments, the freshness sensor further includes a communication means housed in the cap and configured to transmit data from the sensor element to a user. In some embodiments, the communication means is a RFID chip. In other embodiments, the communication means is a NFC chip.

In some embodiments, the freshness sensor further includes sensor paper positioned within the cavity and adjacent to the sensor element.

In some embodiments, the sensor element is configured to monitor gaseous volatile biogenic amines. In other embodiments, the sensor element comprises a volatile nitrogen sensor.

In some embodiments, the expansion portion includes a plurality of longitudinal members that are positioned parallel and adjacent to each other when the spike is inserted into the packaging housing, but which bulge outward and separate to define channels between the bulged longitudinal members.

In some embodiments, the spike includes an outer shell having the expansion portion and an inner shell positioned within the outer shell and defining a plurality of perforations in fluid communication with the cavity defined by the spike.

In some particular embodiments, the outer shell of the spike includes an upper portion having a tooth extending away from the lower surface of the cap, and a lower portion defining a first groove and a second groove, the lower portion having the expansion portion. The upper portion and the lower portion are configured to rotate relative to one another such that, upon rotating the upper portion from a first position in which the tooth of the upper portion is located within the first groove to a second position in which the tooth of the upper portion is located within the second groove, the lower portion is pushed away from the lower surface of the cap causing the expansion portion of the outer shell to expand.

In some particular embodiments, the outer shell of the spike includes a removable collar positioned adjacent to the lower surface of the cap and a lower portion having the expansion portion. Upon removing the collar, the lower portion of the outer shell is pushed towards the lower surface of the cap causing the expansion portion of the outer shell to expand.

In some particular embodiments, the outer shell and the inner shell are configured to slide relative to one another, and an inner surface of the outer shell and an outer surface of the inner shell are configured to engage when the outer shell is pushed towards the lower surface of the cap causing the expansion portion of the outer shell to expand.

In some particular embodiments, the outer shell includes a wider upper portion and a narrower lower portion, the narrower lower portion of the outer shell having the expansion portion. The inner shell also includes a wider upper portion and a narrower lower portion, and the narrower lower portion of the outer shell is narrower than the wider upper portion of the inner shell such that, when the outer shell is pushed towards the lower surface of the cap, the wider upper portion of the inner shell forces the narrow lower portion of the outer shell outward.

In some particular embodiments, the inner surface of the outer shell includes a narrowed portion such that, when the outer shell is pushed towards the lower surface of the cap, the inner shell forces the narrowed portion of the outer shell outward.

In accordance with other exemplary embodiments, a freshness sensor made in accordance with the present invention includes a cap housing electronics, an adhesive layer positioned on a lower surface of the cap, and a spike extending away from the lower surface of the cap. The spike includes electrodes in electrical communication with the electronics. The spike is configured for insertion into a packaging housing a perishable product and into the perishable product. The adhesive layer is configured to seal a hole formed in the packaging by the spike. The electrodes are configured to measure bulk phase electrical properties of the perishable product.

In some embodiments, the electrodes are molded into the spike.

In some other embodiments, the spike includes an upper portion adjacent to and affixed to the cap, a lower portion forming a tapered distal end of the spike, and a middle portion extending between the upper portion and the lower portion. In these embodiments, the electrodes are electrical wires extending along an outside of the middle portion of the spike.

In accordance with another aspect of this disclosure, a method for monitoring the freshness of a perishable item includes the steps of inserting a freshness sensor into a packaging of a perishable item and into the perishable product, monitoring the freshness of the perishable product, and transmitting data from the communication means to a receiving unit. The freshness sensor includes a cap housing communication means, the cap remaining outside of the packaging, a spike extending away from a lower surface of the cap, the spike piercing the packaging and perishable product, and an adhesive layer positioned on a lower surface of the cap, the adhesive layer sealing a hole formed in the packaging by the spike.

In some exemplary implementations, the cap further houses a sensor element and the spike includes a cavity defined by the spike which is in fluid communication with the sensor element. The spike further includes an expansion portion, which is expanded after inserting the freshness sensor into the perishable product to form a hollow in the perishable product to thereby allow gas exchange between the hollow and the sensor element by way of the cavity.

In some exemplary implementations, the spike includes electrodes in electrical communication with the communication means, the electrodes configured to measure bulk phase electrical properties of the perishable product.

Further features and advantages of the present invention will become evident to those of ordinary skill in the art after a study of the description, figures, and non-limiting examples in this document.

### BRIEF DESCRIPTION OF THE DRAWINGS

FIG. 1 is a side view of a first exemplary freshness sensor made in accordance with the present invention;

FIG. 2 is a side sectional view of the first exemplary freshness sensor of FIG. 1;

FIG. 3 is an exploded view of the first exemplary freshness sensor of FIG. 1; and

FIG. 4 is side view of the first exemplary freshness sensor of FIG. 1 with the outer shell of the spike rotated to a second position.

FIG. 5A is a side view of a second exemplary freshness sensor made in accordance with the present invention that includes a removable pull tab;

FIG. 5B is a side partial sectional view of the second exemplary freshness sensor of FIG. 5A; and

FIG. 5C is a side partial sectional view of the second exemplary freshness sensor of FIG. 5A with the pull tab removed and the outer shell moved upward to the cap.

FIG. 6A is a perspective sectional view of a third exemplary freshness sensor made in accordance with the present invention that includes a removable pull tab;

FIG. 6B is a perspective sectional view of the third exemplary freshness sensor of FIG. 6A with the pull tab partially removed;

FIG. 6C is a perspective sectional view of the third exemplary freshness sensor of FIG. 6A with the pull tab removed and the outer shell partially moved upward towards the cap; and

FIG. 6D is a perspective sectional view of the third exemplary freshness sensor of FIG. 6A with the pull tab removed and the outer shell moved entirely upward to the cap.

FIG. 7A is a side view of a fourth exemplary freshness sensor made in accordance with the present invention that includes an electrically conductive component molded into the side of the spike;

FIG. 7B is a side sectional view of a fifth exemplary freshness sensor made in accordance with the present invention that includes an electrically conductive component molded into the side of the spike; and

FIG. 7C is an exploded side view of a sixth exemplary freshness sensor made in accordance with the present invention that includes wiring located outside of the spike.

### DESCRIPTION OF EXEMPLARY EMBODIMENTS

The present invention includes a freshness sensor that is punctured through packaging and into a perishable product, such as meat or other perishable item. In some embodiments, the freshness sensor thereafter creates a hollow in the packaging and/or meat or perishable item to facilitate gas exchange between the meat or other perishable item and the sensor element to monitor factors relating to the freshness of the meat or other perishable item, including but not limited to volatile nitrogen and/or gaseous volatile biogenic amines. In other embodiments, the freshness sensor includes electrodes that measure the bulk phase electrical properties of the meat or other perishable item.

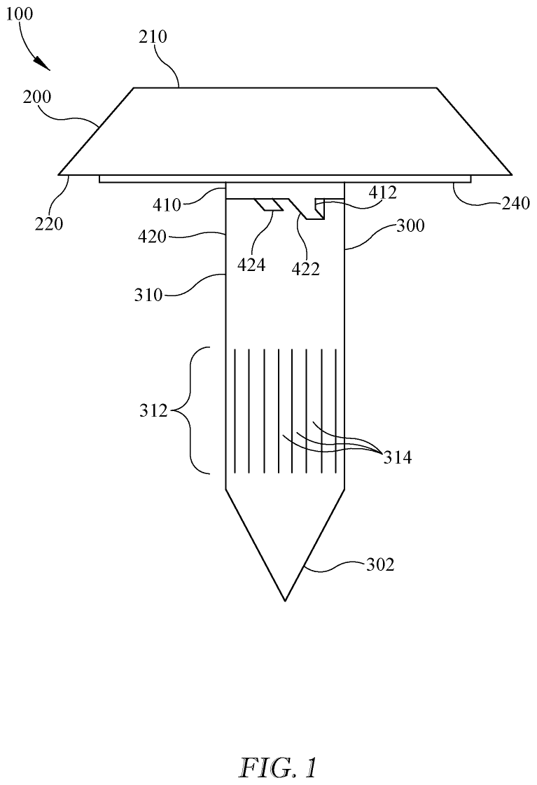

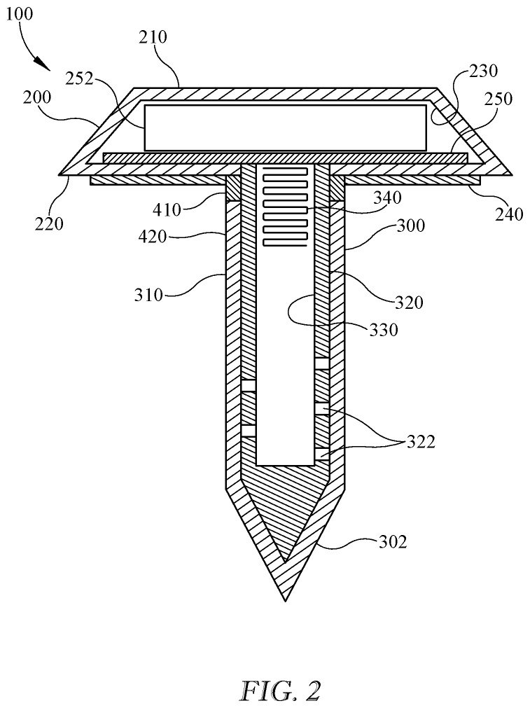

Referring first to FIGS. 1-2, one exemplary freshness sensor 100 made in accordance with the present invention includes a cap 200 having an upper surface 210 and a lower surface 220 opposite the upper surface 210. As shown in FIG. 2, the cap 200 defines a chamber 230 that houses a sensor element 250 as well as electronics 252, as discussed further below. The freshness sensor 100 further includes a spike 300 extending away from the lower surface 220 of the cap 200 and which includes a tapered portion 302 forming a terminal distal end of the spike 300. A self-sealing adhesive layer 240 is further included and is positioned on the lower surface 220 of the cap 200 so as to surround the spike 300 and help secure and maintain placement of the sensor 100, as discussed further below, while also maintaining the hygienic integrity of a package into which the spike 300 may be inserted.

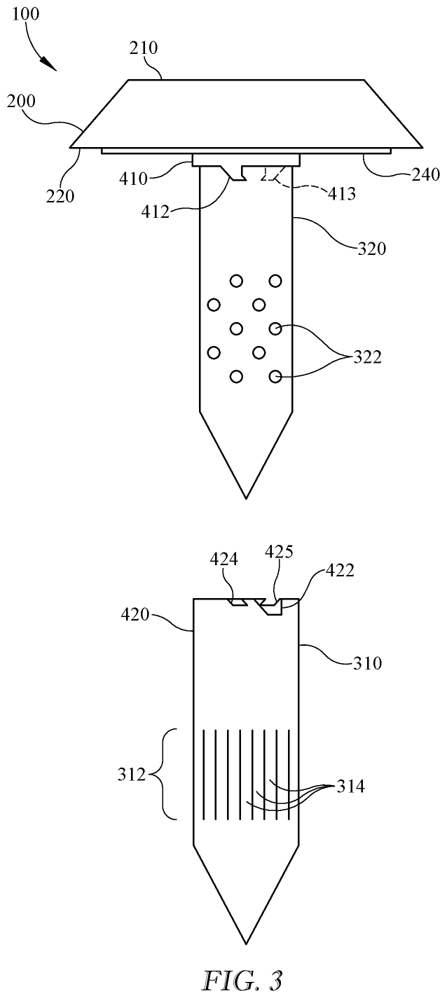

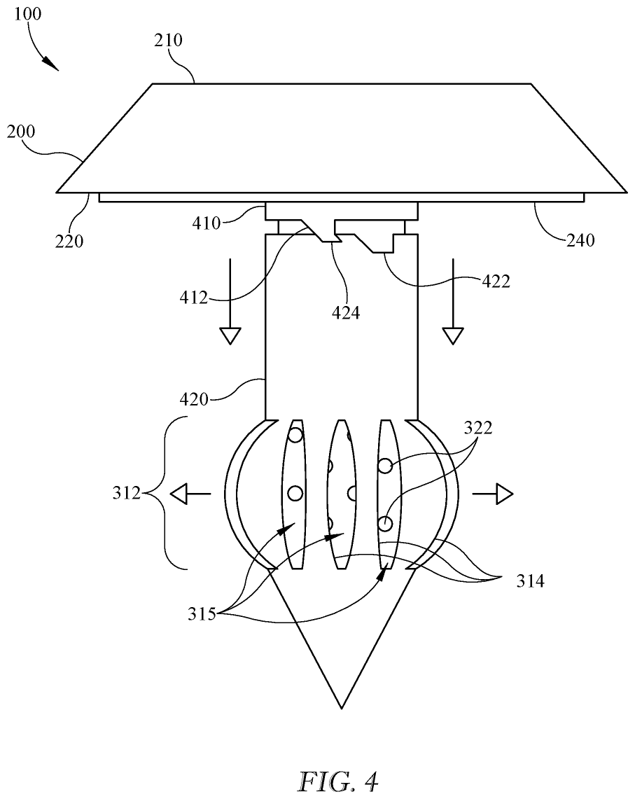

Referring still to FIGS. 1-2 but now also to FIG. 3, the spike 300 includes an outer shell 310 and an inner shell 320 positioned within the outer shell 310. The outer shell 310 includes an expansion portion 312 (shown in FIGS. 1 and 3), which is configured to crack, or otherwise open, upon expansion so as to allow gas exchange, and which is discussed further below with respect to FIG. 4. The inner shell 320 defines a cavity 330 (shown in FIG. 2), which is in fluid communication with the chamber 230 of the cap 200 and the sensor element 250. The inner shell 320 further defines a plurality of perforations 322 that are in fluid communication with the cavity 330 of the inner shell 320. Although illustrated as separate elements in FIG. 2, in this exemplary embodiment, the inner shell 320 is affixed to the cap 200 while the outer shell 310 is, at least in part, moveable in relation to the inner shell 320 and the cap 200.

In particular, and referring now to FIGS. 1-4, the outer shell 310 includes an upper portion 410 that is attached to the cap 200 and a lower portion 420 configured to rotate and slide relative to the upper portion 410 of the outer shell 310, the inner shell 320, and the cap 200. The upper portion 410 of the outer shell 310 includes a tooth 412 extending away from the lower surface 220 of the cap 200. The lower portion 420 of the outer shell 310 then defines a first groove 422 and a slightly smaller second groove 424, which are configured to engage with the tooth 412 of the upper portion 410 as the lower portion 420 is rotated between a first position (shown in FIG. 1) and a second position (shown in FIG. 4). As shown in FIG. 1, when the lower portion 420 is in the first position, the tooth 412 of the upper portion 410 is located within the first groove 422 of the lower portion 420 and there is substantially no gap between the upper portion 410 and the lower portion 420. When initially rotating the lower portion 420 of the outer shell 310, the sloped shape of the tooth 412 and first groove 422 pushes the lower portion 420 away from the upper portion 410. As shown in FIG. 4, upon further rotation, the tooth 412 enters the second groove 424 to prevent any reverse rotation of the lower portion 420. The piston-like movement of the lower portion 420 of the outer shell 310 away from the upper portion 410 of the outer shell 310 then, in turn, causes the expansion portion 312 of the outer shell 310 to expand by shortening the overall length of the lower portion 420 of the outer shell 310. Although the Figures and above description relate to the rotation of the lower portion 420 of the outer shell 310 relative to the upper portion 410 of the outer shell 310, the same movement can be characterized as rotation of the upper portion 410 of the outer shell 310 (and connected cap 200 and inner shell 320) from a first position (shown in FIG. 1) to a second position (shown in FIG. 4) relative to the lower portion 420 of the outer shell 310.

Regardless, as shown in FIGS. 1, 3, and 4, the exemplary expansion portion 312 includes a plurality of longitudinal members 314, which are initially parallel and immediately adjacent to each other (shown in FIGS. 1 and 3), but which bulge outward and separate to define channels 315 between the bulged longitudinal members 314 (shown in FIG. 4). The channels 315 formed in the expansion portion 312 allow gas exchange between the exterior of the freshness sensor 100 and the sensor element 250 by way of the perforations 322 (now exposed through the channels 315) and cavity 330 of the inner shell 320, as discussed further below. According to some embodiments, the plurality of longitudinal members 314 are integrally formed such that, prior to bulging, there are no spaces between the longitudinal members 314 and the outer shell 310 is thus airtight. In some particular embodiments, rather than having longitudinal members, the expansion portion 312 is configured to crack in a manner that allows the expansion portion to expand while also forming cracks large enough to provide sufficient gas exchange through the outer shell 310. Although in the exemplary embodiment shown in FIGS. 1-4, the mechanism for causing the outer shell to bulge is the same as the mechanism for causing the outer shell to become air permeable, in other embodiments, these two mechanism may be separable or completely independent. Likewise, other means of causing the outer shell to bulge and/or selectively allowing gas exchange through the outer shell are also possible without departing from the spirit and scope of the present invention, as discussed further below.

Referring now specifically to FIG. 3, the exemplary freshness sensor 100 includes a second tooth 413 positioned on the opposite side of the upper portion 410 of the outer shell 310. The lower portion 420 of the outer shell 310 includes two additional groove (only one groove 425 shown in FIG. 3) corresponding to the first groove 422 and second groove 424 to engage with this second tooth 413. The particular number and location of these tooth and groove sets is not limited and can be modified depending on the particular design and application of the present invention.

Referring once again to FIG. 2, with respect to the sensor element 250 housed in the cap 200 of the exemplary freshness sensor 100, in some exemplary embodiments, a sensor element is included that is configured to monitor the levels of gaseous volatile biogenic amines which are an indicator of the decay state of meat. In other embodiments, a sensor element is included that is a volatile nitrogen sensor. Of course, other types of sensors can be housed in the cap, including more than one at a time, without departing from the spirit and scope of the present invention.

For example, as shown in FIG. 2, the exemplary freshness sensor 100 further includes sensor paper 340 positioned within the cavity 330 of the inner shell 320 and adjacent to the sensor element 250. The sensor paper 340 is configured to react to one or more gaseous elements in a manner that is detectable by the sensor element 250.

Regardless of the particular sensor element used, the cap 200 of the exemplary freshness sensor 100 further houses electronics 252 which include the circuity and communication means necessary to transmit data from the sensor element to a user. For example, in some embodiments, the electronics 252 include a RFID chip to transmit readings to an RFID reader, smart phone, or other such receiving unit. In still other embodiments, the electronics 252 include a NFC chip for communication with similar receiving unit. In still other embodiments, the electronics 252 include an antenna which can transmit signals at a predetermined frequency for communication with a corresponding signal receiving unit. For additional information and guidance regarding sensors including RFID, NFC, or other transmitters capable of use in accordance with the presently-disclosed subject matter, see, e.g., International Patent Application No. PCT/US21/57083, which is incorporated herein by reference in its entirety.

With further regard to the cap, the shape of the cap 200 is not limited, so long as it is capable of housing the sensor element 250 and electronics 252. For example, when viewed from above, the cap may be circular, square, triangular, or any other shape.

Referring now generally to FIGS. 1-4, in operation, the exemplary freshness sensor 100 described above serves as a monitor to track the freshness of perishable products sold in groceries stores, such as meats, from manufacturing to the consumer's home. The sensor 100 is placed onto existing meat packaging by puncturing the spike 300 through the packaging layer and into the packaged meat. The adhesive layer 240 then seals the hole formed in the packaging layer by the spike 300 by adhering the cap 200 of the sensor 100 to the packaging material. Once again, during insertion of the sensor 100, the plurality of longitudinal members 314 of the expansion portion 312 are positioned parallel and adjacent to each other. Subsequently, the expansion portion 312 is caused to expand (i.e., the plurality of longitudinal members 314 bulge outward and separate to define channels 315 between the bulged longitudinal members 314), as discussed above, which creates a small hollow inside the meat. This hollow improves gas exchange between the surrounding meat and the sensor element 250. Specifically, gases from the meat are able to travel through the channels 315 defined between the longitudinal members 314 in the expansion portion 312 of the outer shell, through the perforations 322 defined by the inner shell 320, through the cavity 330 defined by the inner shell 320, and to the chamber 230 defined by the cap 200 which houses the sensor element 250. The freshness sensor 100 thereby provides a more accurate way to gauge expiration, as it is based off of real time data from the individual packaging biome, rather than an estimate of a larger batch or a statistical approach to freshness dating. Of course, it is also appreciated that while the exemplary sensors described herein are described with particular reference to meats or similar perishable items, the use of the exemplary sensors is not limited to such items as the exemplary sensors can be used to gauge the state of any similarly packaged material, such as flours, grains, bulk commodities, or packaged chemicals.

As previously mentioned, data from the sensor element 250 is transmitted via the electronics 252 to a receiving unit, e.g., a RFID reader or smart phone. According to some implementations, the sensor 100 is scanned in an automated manner by RF signal along the logistics chain including manufacturing facilities, supply chain (e.g., in cargo containers or distribution centers), and in store. If individual sensors are placed on certain meats, it is possible to identify when particular meats have expired or been contaminated, so that instead of disposing of full pallets of potentially expired product, there is a way to sort out and keep sellable product.

According to some other implementations, customers may scan the sensor 100 using an application on their smart phone, which will then identify the product, and give the user an estimate of how much time is left for safe consumption. In this way, a customer will feel comfortable buying the meat, as they trust that they will know for certain when they are able to consume or otherwise use it. Though freshness estimates, e.g., Best-If-Used-By dates are generally accepted, customers still are not sure in the last few days or even a day or two after the estimated date if they might be able to use the product. Furthermore, the actual freshness of the meat could change with unexpected variables, such as the environment the customer keeps their food in, or how long that package was out in the store or home before being refrigerated. Through use of the freshness sensors of the present invention, however, customers know for certain that they are getting an accurate reading on their specific package.

The freshness sensor 100 also provides several contemplated advantages over other types of freshness sensors. As the gas exchange pathways are only formed after the sensor 100 is pierced into the meat, the exemplary freshness sensor 100 avoids meat deformation by shear. The freshness sensor 100 also reduces the likelihood of contamination of the meat from the sensor 100 itself. The exterior of the spike 300 can be easily cleaned without concern of micro pore or complex surface based contamination, and the interior of the sensor 100 remains sealed until after the spike 300 is inserted into the meat. Because the sensor 100 itself creates the hollow inside the meat, there is a consistent gas sensing pocket size. Similarly, the spike style sensor 100 can be used on meat packs that have no gas headspace whatsoever, like ground meat roll packs. In some such instances, the spike style sensor 100 can form a hollow in the packaging instead of, or partially in addition to, forming a hollow in the meat. In any event, with a known gas pocket volume, the freshness calculations are simplified as compared to having to account for variability in meat gas headspace volume. Also, because the sensor 100 is measuring gas samples from the interior of the meat, there is reduced exposure to environment confounding factors such as temperature swings, humidity changes, packaging materials, and atmospheric contamination. Finally, the spike style sensor 100 and adhesive layer 240 can be applied to a variety of meat packs with little to no change to the meat package design or manufacturing process. The adhesive layer 240 seals the hole formed by the spike 300 by adhering the sensor 100 to the packaging material thereby ensuring that external contamination is not permitted to contaminate the meat. In some implementations, the introduction of the spike 300 and sensor 100 into a packaging can be performed using known and generally-accepted aseptic techniques.

As discussed above, in the exemplary freshness sensor 100 shown in FIGS. 1-4, the outer shell 310 is designed such that twisting the outer shell 310 relative to the inner shell 320 causes the piston-like movement that results in the expansion portion 312 of the outer shell 310 to expand. However, according to other embodiments of the present invention, a pull tab is removed to allow for a piston-like movement that similarly causes the outer shell to bulge and become air permeable

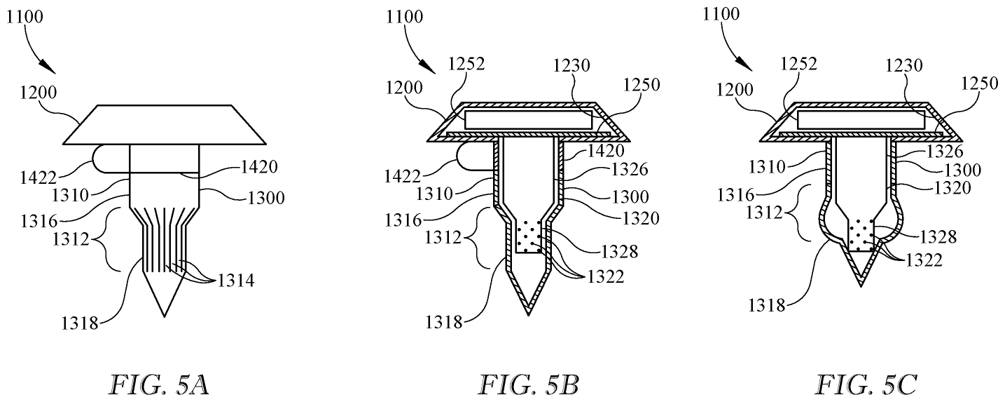

Referring now to FIGS. 5A-5C, a second exemplary freshness sensor 1100 made in accordance with the present invention, includes a cap 1200 similar to the cap 200 described above with respect to FIGS. 1-4, and which, as shown in FIGS. 5B and 5C defines a chamber 1230 that houses a sensor element 1250 and electronics 1252, but the form and operation of the spike 1300 of the freshness sensor 1100 differs.

Also similar to the spike 300 described above with respect to FIGS. 1-4, the exemplary spike 1300 shown in FIGS. 5A-5C includes an outer shell 1310 and an inner shell 1320 positioned within the outer shell 1310. The outer shell 1310 includes an expansion portion 1312, and the inner shell 1320 defines a plurality of perforations 1322 (shown in FIGS. 5B and 5C) that are in fluid communication with a cavity (not shown) of the inner shell 1320 and the chamber 1230 of the cap 200. While the outer shell 310 and inner shell 320 of the sensor 100 described above with respect to FIGS. 1-4 are substantially cylindrical, in the sensor 1100 shown in FIGS. 5A-5C, an inner surface of the outer shell 1310 and an outer surface of the inner shell 1320 are configured to engage when the outer shell 1310 is pushed upwards causing the expansion portion 1312 of the outer shell 1310 to expand.

Specifically, the outer shell 1310 and the inner shell 1320 each have a respective wider upper portion 1316, 1326 and a narrower lower portion 1318, 1328. More specifically, the narrower lower portion 1318 of the outer shell 1310 is narrower than the wider upper portion 1326 of the inner shell 1320. The narrower lower portion 1318 of the outer shell 1310 also overlaps, at least partially, with the expansion portion 1312. The outer shell 1310 further includes a collar 1420 removable via a pull tab 1422. The collar 1420 is positioned adjacent to the cap 1200 and, as shown in FIG. 5C, when removed, allows the outer shell 1310 to be pushed, or otherwise slide, upwards towards the cap 1200. During this piston-like movement of the outer shell 1310, the wider upper portion 1326 of the inner shell 1320 forces the narrower lower portion 1318 of the outer shell 1310 outward. In other words, it causes the expansion portion 1312 of the outer shell 1310 to expand. Similar to the outer shell 310 described above with respect to FIGS. 1-4, as shown in FIG. 5A, the expansion portion 1312 includes a plurality of longitudinal members 1314 which are initially integrally formed such that, prior to bulging, there are no spaces between the longitudinal members 1314 and the outer shell 1310 is airtight. However, once the expansion portion 1312 bulges outward, channels (not shown) are formed between the bulged longitudinal members 1314 to allow gas exchange between the exterior of the freshness sensor 1100 and the sensor element 1250 by way of the perforations 1322 and cavity (not shown) of the inner shell 1320, as discussed above.

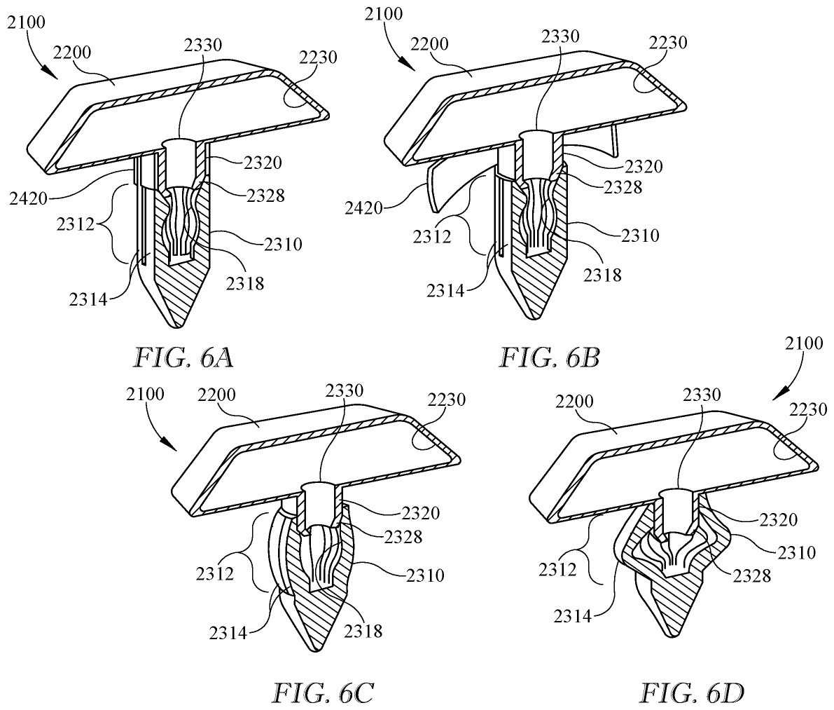

Referring now to FIGS. 6A-6D, a third exemplary freshness sensor 2100 made in accordance with the present invention operates in substantially the same manner as the second exemplary freshness sensor 1100, but the form and function of both the outer shell 2310 and inner shell 2320 differ. Similar to the second exemplary freshness sensor 1100, the outer shell 2310 of the third exemplary freshness sensor 2100 includes a removable collar 2420 located immediately adjacent to the cap 2200 which, as shown in FIGS. 6C and 6D, when removed, allows the outer shell 2310 to be pushed, or otherwise slide, upwards towards the cap 2200. However, the shape of the outer shell 2310 shown in FIGS. 6A-6D is different from the outer shell 1310 described above with respect to FIGS. 5A-5C. Specifically, the outer shell 2310 shown in FIGS. 6A-6D has a contoured inner surface with a narrowed portion 2318 (shown in FIGS. 6A and 6B) which overlaps with the expansion portion 2312. Furthermore, the inner shell 2320 shown in FIGS. 6A-6D is substantially shorter than the inner shell 1320 shown in FIGS. 5A-5C and has a tapered leading edge 2328. In some implementations, this process separates the forces required for insertion of the sensor in the bulk meat from the forces required to cause the expansion, thereby avoiding accidental expansion of the sensor.

As shown in FIG. 6C, after the collar 2420 is removed, the outer shell 2310 slides upwards towards the cap 2200 and the leading edge 2328 of the inner shell 2320 forces the narrowed portion 2318 of the outer shell 2310 outward causing the expansion portion 2312 to begin to bulge. Then, as shown in FIG. 6D, further movement of the outer shell 2310 upwards towards the cap 2200 continues the bulging of the expansion portion 2312. Similar to the outer shells 310, 1310 described above, the expansion portion 2312 shown in FIGS. 6A-6D includes a plurality of longitudinal members 2314 which are initially integrally formed such that, prior to bulging, there are no spaces between the longitudinal members 2314 and the outer shell 2310 is airtight. However, once the expansion portion 2312 bulges outward, channels (not shown) are formed between the bulged longitudinal members 2314 to allow gas exchange between the exterior of the freshness sensor 2100 and a sensor element (not shown) housed in the chamber 2230 of the cap 2200 by way of the cavity 2330 of the inner shell 2320. Of course, with the shorter inner shell 2320, there is no need for the perforations to facilitate gas exchange into the cavity 2330 of the inner shell 2320.

Each of the freshness sensors 100, 1100, 2100 described above are designed for use with a gas sensor element. However, in other embodiments of the present invention, the freshness sensor includes an external electrically conductive component designed to measure bulk meat phase conductivity testing to determine the degree of decomposition

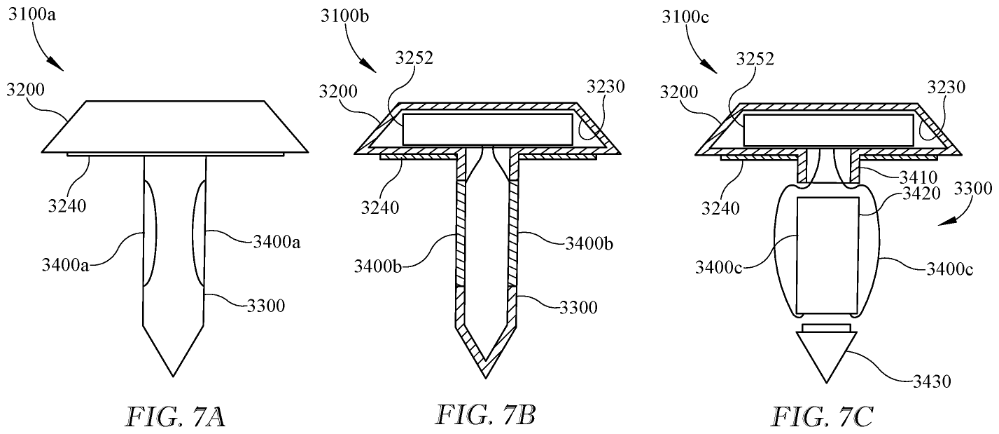

Referring now to FIGS. 7A-7C, three similarly configured freshness sensors 3100a, 3100b, 3100c made in accordance with the present invention include a cap 3200 defining a chamber 3230 that houses electronics 3252 (only shown in FIGS. 7B and 7C), spike 3300, and adhesive layer 3240 similar to the other embodiments described above. In the freshness sensors 3100a and 3100b shown in FIGS. 7A and 7B, the spike 3300 includes electrodes 3400a, 3400b, molded into the body of the spike 3300 whereas in the freshness sensor 3100c shown in FIG. 7C, the spike 3300 includes electrodes in the form of electrical wiring 3400c held in place between an upper portion 3410 of the spike 3300, a middle portion 3420 of the spike 3300, and a lower portion 3430 of the spike 3300 such that the wirings 3400c are located along an outside of the middle portion 3420 of the spike 3300. In all three freshness sensors 3100a, 3100b, 3100c, the respective electrically conductive component, 3400a, 3400b, 3400c is in electrical communication with the electronics 3252 which includes a communication means, e.g., a RFID chip, housed in the chamber 3230 of the cap 3200 of the sensor 3100a, 3100b, 3100c. In operation, the sensors 3100a, 3100b, 3100c shown in FIGS. 7A-7C are placed onto existing meat packaging by puncturing the spike 3300 through the packaging layer and into the packaged meat with the adhesive layer 3240 sealing the hole formed by the spike 3300 by adhering the sensor 3100a, 3100b, 3100c to the packaging material in substantially the same manner as described above. However, by measuring the change in meat bulk phase electrical properties to determine decomposition, many of the challenges of a gas sensor type sensor are avoided.

Lastly, further provided in accordance with some embodiments of the presently-disclosed subject matter are methods for monitoring the freshness of a perishable item that make use of the sensors described herein. In some implementations, a method for monitoring the freshness of a perishable item is provided that comprises the steps of inserting an exemplary sensor of the present invention into the packaging of a perishable; and monitoring the freshness of the perishable product. In some implementations, the exemplary methods further include aseptic or antiseptic handling techniques or include or make use of outer packaging. In some implementations, such monitoring of the freshness includes transmitting data from the sensor element included in an exemplary freshness sensor to a receiving unit, and identifying, via the receiving unit, the freshness of the perishable item.

One of ordinary skill in the art will recognize that additional embodiments and implementations are also possible without departing from the teachings of the present invention or the scope of the claims which follow. This detailed description, and particularly the specific details of the exemplary embodiments disclosed herein, is given primarily for clarity of understanding, and no unnecessary limitations are to be understood therefrom, for modifications will become apparent to those skilled in the art upon reading this disclosure and may be made without departing from the spirit or scope of the claimed invention.

What is claimed is:

1. A freshness sensor comprising:
a cap housing a sensor element; and
a spike extending away from a lower surface of the cap, the spike including
a cavity defined by the spike, the cavity in fluid communication with the sensor element, and
an expansion portion, the expansion portion configured to expand after the spike is inserted into a packaging housing a perishable product and to form a hollow in the packaging or perishable product to thereby allow gas exchange between the hollow and the sensor element by way of the cavity.

2. The freshness sensor of claim 1, further comprising an adhesive layer positioned on the lower surface of the cap.

3. The freshness sensor of claim 1, wherein the spike has a tapered portion forming a terminal distal end of the spike.

4. The freshness sensor of claim 1, further comprising a communication means housed in the cap and configured to transmit data from the sensor element to a user.

5. The freshness sensor of claim 4, wherein the communication means is a RFID chip.

6. The freshness sensor of claim 4, wherein the communication means is a NFC chip.

7. The freshness sensor of claim 1, further comprising sensor paper positioned within the cavity and adjacent to the sensor element.

8. The freshness sensor of claim 1, wherein the sensor element is configured to monitor gaseous volatile biogenic amines.

9. The freshness sensor of claim 1, wherein the sensor element comprises a volatile nitrogen sensor.

10. The freshness sensor of claim 1, wherein the expansion portion includes a plurality of longitudinal members that are positioned parallel and adjacent to each other when the spike is inserted into the packaging housing, the longitudinal members further configured to bulge outward and separate to define channels between the bulged longitudinal members.

11. The freshness sensor of claim 1, wherein the spike includes an outer shell and an inner shell positioned within the outer shell, the outer shell having the expansion portion, and the inner shell defining a plurality of perforations in fluid communication with the cavity defined by the spike.

12. The freshness sensor of claim 11, wherein the outer shell of the spike includes an upper portion having a tooth extending away from the lower surface of the cap, and a lower portion defining a first groove and a second groove, the lower portion having the expansion portion;
wherein, the upper portion and the lower portion are configured to rotate relative to one another; and
wherein, upon rotating the upper portion from a first position in which the tooth of the upper portion is located within the first groove to a second position in which the tooth of the upper portion is located within the second groove, the lower portion is pushed away from the lower surface of the cap causing the expansion portion of the outer shell to expand.

13. The freshness sensor of claim 11, wherein the outer shell of the spike includes a removable collar positioned adjacent to the lower surface of the cap and a lower portion, the lower portion having the expansion portion; and
wherein, upon removing the collar, the lower portion of the outer shell is pushed towards the lower surface of the cap causing the expansion portion of the outer shell to expand.

14. The freshness sensor of claim 11, wherein the outer shell and the inner shell are configured to slide relative to one another; and
wherein an inner surface of the outer shell and an outer surface of the inner shell are configured to engage when the outer shell is pushed towards the lower surface of the cap causing the expansion portion of the outer shell to expand.

15. The freshness sensor of claim 14, wherein the outer shell includes a wider upper portion and a narrower lower portion, the narrower lower portion of the outer shell having the expansion portion;
wherein the inner shell includes a wider upper portion and a narrower lower portion; and
wherein the narrower lower portion of the outer shell is narrower than the wider upper portion of the inner shell such that, when the outer shell is pushed towards the lower surface of the cap, the wider upper portion of the inner shell forces the narrow lower portion of the outer shell outward.

16. The freshness sensor of claim 14, wherein the inner surface of the outer shell includes a narrowed portion such that, when the outer shell is pushed towards the lower surface of the cap, the inner shell forces the narrowed portion of the outer shell outward.

17. A freshness sensor comprising:
a cap housing electronics;
an adhesive layer positioned on a lower surface of the cap; and
a spike extending away from the lower surface of the cap, the spike including electrodes in electrical communication with the electronics;
wherein, the spike is configured for insertion into a packaging housing a perishable product and into the perishable product, the adhesive layer is configured to seal a hole formed in the packaging by the spike, and the electrodes are configured to measure bulk phase electrical properties of the perishable product.

18. The freshness sensor of claim 17, wherein the electrodes are molded into the spike.

19. The freshness sensor of claim 17, wherein the spike includes an upper portion adjacent to and affixed to the cap, a lower portion forming a tapered distal end of the spike, and a middle portion extending between the upper portion and the lower portion;
wherein the electrodes are electrical wires extending along an outside of the middle portion of the spike.

20. A method for monitoring the freshness of a perishable item, comprising:
inserting a freshness sensor into a packaging of a perishable item and into the perishable product, the freshness sensor including
a cap housing communication means, the cap remaining outside of the packaging,
a spike extending away from a lower surface of the cap, the spike piercing the packaging and perishable product, and
an adhesive layer positioned on a lower surface of the cap, the adhesive layer sealing a hole formed in the packaging by the spike;
monitoring the freshness of the perishable product; and
transmitting data from the communication means to a receiving unit.

21. The method of claim 20, wherein the cap further houses a sensor element and the spike includes
a cavity defined by the spike, the cavity in fluid communication with the sensor element, and
an expansion portion;
wherein, after inserting the freshness sensor into the perishable product, the expansion portion is expanded to form a hollow in the perishable product to thereby allow gas exchange between the hollow and the sensor element by way of the cavity.

22. The method of claim 20, wherein the spike includes electrodes in electrical communication with the communication means, the electrodes configured to measure bulk phase electrical properties of the perishable product.
